(19) United States
(12) Patent Application Publication
(10) Pub. No.: US 2023/0058373 A1
(43) Pub. Date: Feb. 23, 2023

(54) PH SENSOR DEVICE INTENDED TO BE INSERTED INTO THE GROUND, METHOD FOR MEASURING PH, IN PARTICULAR FOR CATHODIC PROTECTION

(71) Applicants: GRTGAZ, BOIS-COLOMBES (FR); CENTRE NATIONAL DE LA RECHERCHE SCIENTIFIQUE, PARIS (FR); SORBONNE UNIVERSITE, PARIS (FR)

(72) Inventors: Catherine DEBIEMME-CHOUVY, SAINT-MAUR-DES-FOSSES (FR); Ahmed FAKHRY, CHOISY-LE-ROI (FR); Elisabeth FLEURY, ERMONT (FR)

(21) Appl. No.: 17/790,075

(22) PCT Filed: Dec. 19, 2020
(86) PCT No.: PCT/FR2020/052575

§ 371 Date: Jun. 29, 2022

(30) Foreign Application Priority Data
Dec. 30, 2019 (FR) 1915721

(51) Int. Cl. C23F 13/04; G01N 33/24; G01N 27/416; G01N 27/30

(52) U.S. Cl. CPC C23F 13/04; G01N 33/24; G01N 27/4167; G01N 27/302

(57) ABSTRACT

A pH sensor device, intended to be inserted into the ground in order to be in contact with a region containing a fluid from the ground, for which fluid the pH is desired to be known, comprising:
a pH sensor comprising a surface covered in a polymer material configured to be in contact with the region containing a fluid when the device is inserted into the ground,
a metal element having a surface configured to be in contact with the region containing the fluid when the device is inserted into the ground,
electrical connection means connected at least to the metal element and configured to receive a cathodic-protection electrical potential for the metal element.

The invention also relates to a method for measuring pH that can be used for cathodic protection.

### TECHNICAL FIELD

The present invention concerns the field of determining ground characteristics. In particular, it concerns determining the pH of a region of the ground in which a pipe can be installed and for which cathodic-protection is implemented.

### PRIOR ART

Pipes which are buried in the ground and which transports fluids (typically gas) are generally made of steel with a composition which makes corrosion possible. This corrosion is generally associated with the pH of the ground, or more precisely with the pH of a fluid contained in the ground (for example water).

Diagrams, called Pourbaix diagrams, are known for different materials. These diagrams allow a material to be located in different zones which illustrate the corrosion risk (typically corrosion, passivation, immunity) as a function of two parameters: the pH of the fluid in contact with the material and the electrical potential applied to the material. It follows that an electrical potential is generally applied to pipes so that they can be located in the zone in which corrosion rate is reduced to a value sufficiently low to meet the standards in force. Methods in which an electrical potential is applied to a pipe are called cathodic-protection methods.

Standard NF EN ISO 15589-1 requires the electrical potential of buried metal structures to be maintained below 0.85V/Cu—CuSO₄. In practice, the electrical potential of pipes is maintained at approximately 1V/Cu—CuSO₄.

It is understood that the pH of fluids contained in the ground can vary as a function of many parameters, and this pH can also be influenced by the cathodic protection itself.

Therefore, knowledge of the value of the pH of the fluid which surrounds a pipe is critical.

It is not possible to carry out an excavation in order to reach a buried pipe and measure a value of the pH, given the cost that this represents.

Placing pH indicator paper in the ground close to pipes, but not at the same depth, has been proposed. This method does not give a precise value of the pH of the ground, but only an order of magnitude. In addition, it is subjective because it depends on the assessment of the colour of the indicator paper by the operator, which colour may be distorted by the colour of the ground itself. Finally, a measurement in which pH indicator paper is used is temporary and does not provide a continuous knowledge of the pH.

The so-called leachate method proposes sampling a portion of the soil, diluting it and measuring the pH of the resulting solution. This method provides an approximation of the pH, but this is not sufficiently precise since the medium is diluted, which necessarily modifies the actual value of the pH of the medium. This method also as the disadvantage that it cannot be carried out in a laboratory using a conventional pH probe (which generally only functions in a liquid medium).

Also known is a pH sensor marketed by the American company M. C. Miller CO. Inc. which uses an antimony-based electrode. This pH sensor has some disadvantages. Firstly, antimony is toxic and carcinogenic, which poses problems of the safety for the users. Further, this sensor delivers an open-circuit potential value which is correlated with a pH value, but this correlation is only possible on the basis of a calibration carried out beforehand in an unknown medium. For this reason, an uncertainty remains for this measurement because the pH varies from one medium to another, depending on the species present in the medium.

Document WO2008031453 describes a method for determining the passivation properties of a mixture containing at least two components (cement and water). In that document, the passivation measurement on the surface of a reference steel makes it possible to detect the presence of water or aggressive substances, a chlorine content that is too high or a pH that is too low.

The invention aims to overcome at least some of the above-mentioned disadvantages.

### DISCLOSURE OF THE INVENTION

To this effect, the invention proposes a pH sensor device, intended to be inserted into the ground in order to be in contact with a region containing a fluid from the ground, for which fluid the pH is desired to be known, comprising:
a pH sensor comprising a surface comprising a polymer material capable of attracting protons and arranged so that the polymer material is in contact with said region containing a fluid when the device is inserted into the ground,
a metal element having a surface configured to be in contact with the region containing the fluid when the device is inserted into the ground,
electrical connection means connected at least to the metal element and configured to receive a cathodic-protection electrical potential for the metal element.

The inventors of the present invention have observed that it is possible to use polymer materials to produce pH sensors, and that by combining these sensors with a metal element receiving an electrical potential, a shallow-depth measurement can be performed in an environment close to that of a buried pipe which itself also receives a cathodic-protection electrical potential. The pH measurement by the sensor thus clearly illustrates a pH measurement which could be made in the close vicinity of a pipe (for example at several tens of centimetres from a pipe)

Furthermore, the use of polymers enables a robust sensor to be obtained, which can be used in the ground while having a good service life. Further, these polymers do not have the disadvantages of sensors containing antimony which are dangerous for users. Polymers are preferably chosen containing groups that can attract protons, so that the electrical potential at the surface will be affected.

Typically, the electrical potential is an electrical potential having a value which could be applied to a pipe (for example less than −0.85V/Cu—CuSO₄).

Electrical connection means which include a cable can be used to apply this electrical potential.

The surface comprising a polymer material capable of attracting protons can also be arranged so as to at least partially cover another surface of the pH sensor, for example a surface of another element made of metal which is conductive and able to transmit the signal resulting from the presence of protons on the surface comprising a polymer material.

According to a particular embodiment, the polymer contains one or more amino groups (in their molecular chain).

The inventors have observed that amino groups function well for the formation of pH sensors.

By way of indication, the polymer may be a conductive polymer.

According to a particular embodiment, the polymer containing amino groups is chosen from the group comprising polypyrrole and poly(3,4 ethylenedioxythiophene).

These two polymer materials are conductors.

According to a particular embodiment, the device comprises a body provided with at least a first opening configured to allow, when the device is inserted into the ground, the fluid contained in said region to be in contact with said surface comprising a polymer material and with said surface of the metal element, and at least one second opening configured to receive said electrical connection means.

This body can comprise a polymer-based material that will be chosen for its mechanical properties and its resistance to fluids with which the body may come into contact in the ground.

By way of indication, the first opening can be the lower opening of the body, oriented downwards when the device is inserted into the ground. The second opening is oriented upwards in order to allow passage for the electrical connection means.

According to a particular embodiment, said surface comprising a polymer material and said surface of the metal element are arranged recessed from an outer surface of the body where the openings are formed.

This recess enables these two surfaces to be protected.

For example, these two surfaces may be recessed by a millimetre.

According to a particular embodiment, the device comprises first means for maintaining the sealing arranged at the interface between, on the one hand, said surface comprising a polymer material and said surface of the metal element and, on the other hand, the body, in order to prevent the fluid contained in said region from penetrating inside the body.

It has been observed that it is particularly important to maintain the sealing of the device once it is in contact with the fluid present in the ground. The means for creating the sealing may comprise, for example, one or more seals.

According to a particular embodiment, the device comprises second means for maintaining the sealing arranged at the interface between, on the one hand, the electrical connection means and, on the other hand, the body, in order to prevent the fluid contained in said region from penetrating inside the body.

In the same way, these second means for creating sealing can comprise one or more seals and/or a cable gland.

According to a particular embodiment, the pH sensor and/or the metal element are detachable from the body.

It can be particularly useful to be able to change these elements independently of other elements of the device.

By detachable, it is meant that the pH sensor and/or the metal element can be removed either without tools (for example if they are attached by snap-fitting) or with a tool (for example if they are attached by screwing).

The electrical connection means can be connectable and disconnectable means, for example comprising plugs.

According to a particular embodiment, said surface comprising a polymer material and said surface of the metal element are spaced apart by a strip having a thickness between 0.5 and 1.5 millimetres, for example equal to 1 millimetre.

This strip makes it possible to avoid any electrical contact between the metal element and the polymer surface. Preferably, a strip is used having a thickness of 1 millimetre so that the presence of the metal element can clearly illustrate the environment in which a buried pipe is located.

According to a particular embodiment, said surface of the metal element has an annular shape and said surface comprising a polymer material is arranged so as to be surrounded by said surface of the metal element.

It has been observed by the inventors of the present invention that this arrangement, wherein the surface comprising a polymer material surrounded by the surface of the metal element clearly illustrates the proximity of a sensor with a pipe, and enables, in particular, a better sensitivity to be obtained to the changes in pH of the medium in contact with the surface of the polymer material.

According to a particular embodiment, said surface of the metal element has an area greater than or equal to at least 1 cm² and said surface comprising a polymer material has an area greater than or equal to at least 0.07 cm².

The invention also proposes a method for measuring the pH of a fluid contained in a region of the ground, comprising:
inserting a device, such as that defined above, into the ground so that said surface of the metal element and said surface comprising a polymer material are in contact with the fluid contained in a region of the ground,
applying a cathodic-protection electrical potential to the metal element via the electrical connection means, and
measuring the pH by means of the pH sensor.

This method will preferably be implemented in the vicinity of an actually buried pipe, for example above this pipe.

For example, this method will be implemented close to a station for measuring (or tapping) the electrical potential of the pipe, generally arranged above the pipes. These measurement stations are of course placed on the surface of the ground. In this case, the invention advantageously allows the same electrical potential to be applied to the metal element as that which is applied to the pipe (it is tapped directly on the pipe). Thus, an even more realistic measurement of the pH at the level of the buried pipe is obtained.

According to a particular embodiment, the method comprises:
implementing the method for measuring pH as defined above for a region of the ground located above a pipe,
determining a new value of electrical potential to be applied to said pipe on the basis of the result of said pH measurement.

Thus the invention enables the cathodic-protection of buried pipes to be improved, without it being necessary to implement measurements directly at the level of the buried pipes.

By way of indication, the determining of the new value of electrical potential can be performed partially automatically, for example by a computer program executed by a processor of the device or connected to the device, and on the database stored in a memory of the device or of another device such as a Pourbaix diagram.

For example, this computer program can deliver a range of possible new values, or a single possible value.

### BRIEF DESCRIPTION OF THE FIGURES

Other features and advantages of the present invention will become apparent from the description given below, with reference to the appended drawings which illustrate an exemplary embodiment that is in no way limiting. In the figures:

FIG. 1 is an example of a Pourbaix diagram.

FIG. 2 is a sectional view of a first device example.

FIG. 3 is a view from below of a first device example.

FIG. 4 is an exploded view of the first device example.

FIG. 5 is a sectional view of a second device example.

FIG. 6 is a view from below of the second device example.

FIG. 7 is an exploded view of the second device example.

FIG. 8 illustrates electrical potential tapping stations used in an example above a pipe.

### DESCRIPTION OF THE EMBODIMENTS

Devices will now be described which enable pH measurements of fluids contained in the ground, which can be inserted into the ground at shallow depth and which obtain a value which clearly illustrates that which could be measured close to a more deeply buried pipe.

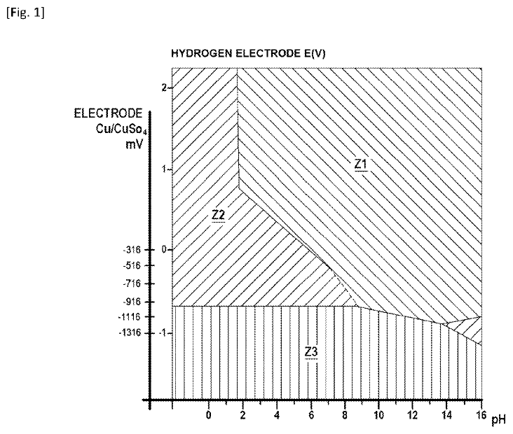

FIG. 1 is an example of a Pourbaix diagram, which shows in which zone a metal in contact with a fluid is located, as a function of the pH of this fluid and of the electrical potential applied to the metal.

There are, in particular, three main zones: the passivation zone Z1, the corrosion zone Z2, and the immunity zone Z3. In general, an electrical potential is applied in order that a metal is located in a zone where the risk of corrosion is low or even where the rate of corrosion is low.

In the event of variation (low or high) of the pH, it can therefore be useful to determine a new value of electrical potential to be applied, for example automatically by reading from a chart based on a Pourbaix diagram.

It has also been observed that the application of an electrical potential on a metal can have an effect on the value of the pH.

The use of a simple pH sensor at shallow depth is therefore not sufficient to determine the pH value of the fluid in contact with a surrounded pipe.

It should be noted that, in general, pipes are buried at a minimum depth of 80 cm. With the invention which will be described below, a measurement can be performed at shallow depth, typically of order 20 to 50 cm, but nevertheless resulting in a pH value which clearly illustrates that which it would be possible to measure close to the pipe, for example at a distance less than ten centimetres.

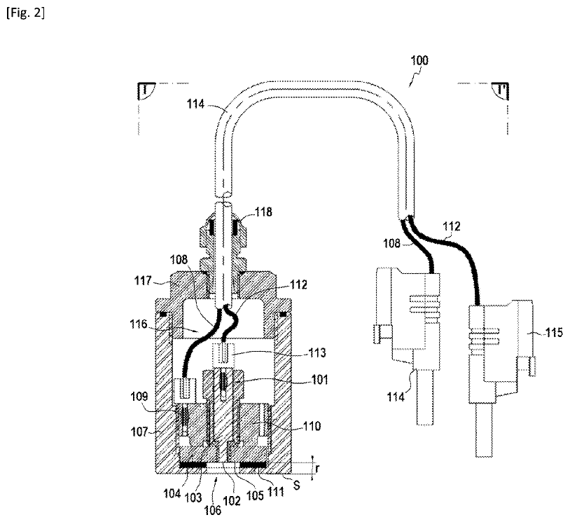

In FIG. 2, a pH sensor device 100 is shown in cross-section. This device is intended to be inserted into the ground, for example at a depth of order 20 to 30 cm, and advantageously above a pipe for which it is desired to check the state of corrosion. More precisely still, this device can be inserted into the ground close to an electrical potential measurement (or potential tapping) station of the pipe that is generally arranged above the pipes.

This device 100 includes a pH sensor 101 comprising an electrode having a surface 102 which contains polypyrrole located at the bottom in the figure. The invention is nevertheless not limited to the use of polypyrrole and also relates to the use of other conductive polymers such as, for example, poly(3,4 ethylenedioxythiophene), also designated by the name PEDOT.

The invention is nevertheless not limited to the use of these two materials and can be implemented with any polymer capable of attracting protons, for example polymers comprising one or more amino groups.

Obtaining a layer of polypyrrole can be achieved following the method described in the thesis “Synthèse par voie électrochimique de nanostructures de polymères conducteurs sans emploi d'une matrice support” (Electrochemical synthesis of conductive polymer nanostructures without the use of a support matrix] (Ahmed Fakhry, 2014, Université Pierre et Marie Curie).

More specifically, a polypyrrole film can be formed by electrochemical means on a support which can be made of stainless steel, platinum, gold, etc. This support is given reference sign 103 in the figure and forms, with the surface 102, an electrode of the pH sensor 101. Thus, the polypyrrole covers the surface of the support in order to form the outer surface 102.

The formation or synthesis is carried out in a three-electrode cell containing a solution with a pyrrole monomer, a dopant such as perchlorate (ClO₄⁻) and a support electrolyte, for example K₂HPO₄.

This makes it possible to obtain polypyrrole nanostructures which are oriented towards the outside of the sensor, downwards in the figure.

It should be noted that polypyrrole is a conductive polymer, the molecular structure of which contains so-called “amino” groups which have an affinity for protons present in the medium in contact with the polypyrrole (for example a fluid). The reaction of protons with the “amino” groups thus creates an excess charge density local to the surface of the electrode containing the polypyrrole. The response measured by a variation in electrical potential can thus be considered as being a behaviour controlled by a surface reaction which is produced on the polymer film.

It should also be noted that polypyrrole is used as material for equipping a surface of a pH sensor. Polypyrrole is an environmentally friendly material that is easy to synthesise and inexpensive.

Furthermore, it should be noted that it is possible to implement a calibration step of the resulting sensor. This calibration can be implemented in a liquid medium or even in the ground in the presence of a liquid. In the ground, the electrode is placed in a region for which the pH is known, in order to measure the free potential or open circuit potential. By repeating this measurement for different pH values, a calibration is obtained for the electrode and the sensor, and the sensor can be used in any media.

The device 100 also includes a metal element 104, typically formed from a material which is preferably the same as that of the pipe for which it is desired to perform a measurement.

The metal element can also be designated as a metal coupon or a gravimetric coupon by a person skilled in the art.

This element includes an annular surface 105 which surrounds the surface 102 described above.

Indeed, the surfaces 105 and 102 are both arranged towards the bottom in the figure in the middle of a first opening 106 of a body 107 in which the pH sensor and the metal element are placed. The body can be made of a polymer material and be, for example, made of polypropylene. Here, the body 107 has a substantially circular cylindrical form, but other shapes are possible.

Both surfaces 105 and 102 are therefore left free so that if the device is inserted into the ground, these surfaces will be in contact with the fluid contained in the ground, thus the surface 102 will be used for the purposes of pH measurement.

In order that this measurement can clearly illustrate the value of the pH that could be measured directly in the vicinity of a buried pipe to which an electrical potential has been applied, an electrical potential is also applied to the metal element 104. For this reason, the surface 105 will affect the pH in the same way as the proximity of the pipe, which makes the measurement by the sensor 101 very close to that which could be made in the proximity of the buried pipe.

It should be noted that the surfaces 102 and 105 are recessed from the surface S of the body, this recess having a depth denoted by r of order one millimetre, in order to protect the two surfaces.

In order to apply this electrical potential, electrical connection means are used comprising:
a cable 108 for bringing an electrical potential to the metal element 104,
a plug 109 mounted at the end of cable which is in the device, and
a connector 110, made for example of brass covered with nickel, pressed against the metal element 104 by screwing.

It should be noted that in order to avoid the fluid penetrating inside the body, a seal 111 is arranged around the surface 105, between the metal element 104 and a portion of the body 107 against which the seal is held. The seal 111 forms first means for maintaining the sealing.

Electrical connection means are also used in order to read the pH value delivered by the sensor 101, said means comprising:
a cable 112, and
a plug 113 inserted into the sensor 101.

The two cables 108 and 112 are assembled in a sleeve 114 in order to form a cable harness, and the two ends of these cables 108 and 112 which are not in the body 107 are respectively connected to plugs 114 and 115, for example plugs according to standard CEI 61010.

The two cables 108 and 112 assembled in the sleeve 114 leave the body 107 via a second opening 116 of the body, and the body is closed by a stopper 117, made for example from polypropylene, and open to allow the two cables 108 and 112 to pass assembled in the sleeve 114 through a cable gland 118 which forms the second means for maintaining the sealing.

FIG. 3 is a view from below of the device 100 described with reference to FIG. 2. This figure shows the surface 102 made of polypyrrole which has a circular shape and which is placed at the centre of the device. The surface 102 is surrounded by the annular-shaped surface 105.

This figure also shows the sectional plane I-I′ corresponding to FIG. 2.

By way of indication, the surface 102 has an area equal to 0.07 cm². The surface 105 has an area equal to 1 cm². Larger areas are possible for these two surfaces.

Advantageously, an annular-shaped strip 119 separates the surfaces 102 and 105. This strip has a thickness e.

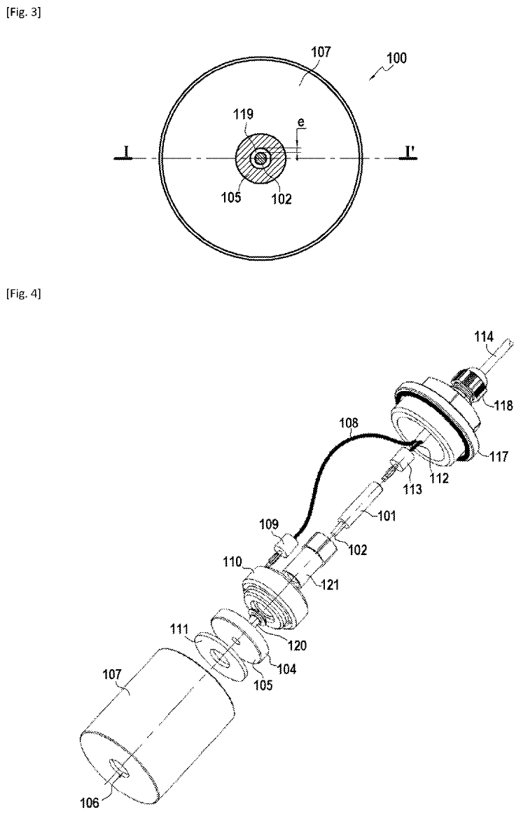

FIG. 4 is an exploded view of the device 100 described with reference to FIGS. 2 and 3. This figure also shows that the sensor 101 is inserted in a bushing 120 at its end comprising the surface 102, and in an insulating attachment flange 121 at the connector 110.

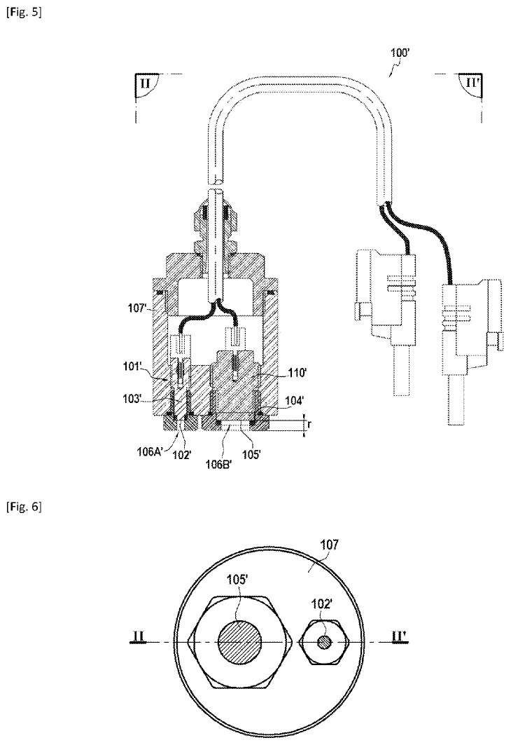

FIG. 5 is a sectional view of a device 100′, which differs from that of FIGS. 2 to 4 in that the surface of the metal element and the surface comprising the polypyrrole are adjacent without that made of metal surrounding that comprising polypyrrole. For the sake of conciseness, the elements which are identical to those described with reference to FIGS. 2 to 4 are not described in relation to the device 100′.

More specifically, the device 100′ includes a pH sensor 101′ with a surface 102′ comprising polypyrrole. The sensor 101′ is identical to the sensor 101 except that it is placed against a wall of the body 107′ of the device 100′, and inserted directly in the body 107.

The device 100′ also includes a metal element 104′ having a surface 105. The metal element 104′ is arranged diametrically opposite the pH sensor 101′. A nickel-covered brass connector 110′ is also used to receive the electrical potential in the same way as by using connector 110 of the device 100. The connector 110′ is mounted in the body 107′. Connectors comprising other materials could be used, for example other conductive connectors.

Here, the body 107 has two first openings 106A′ and 106B′ which receive the surfaces 102′ and 105′ respectively.

The surfaces 102′ and 105′ are recessed from the surface S′ of the body, with a recess having a depth r of order one millimetre.

FIG. 6 is a view from below of the device 100′ described with reference to FIG. 5. This figure shows the polypyrrole surface 102′ which has a circular shape and is located to the right in the figure. The surface 102′ is adjacent to the surface 105′ in the form of a disc and which is located to the left in the figure.

This figure also shows the sectional plane II-II′ corresponding to FIG. 5.

FIG. 7 is an exploded view of the device 100′ described with reference to FIGS. 5 and 6. This figure also shows that the sensor 101′ is inserted in a bushing 120 at its end comprising the surface 102. Here, the bushing is partially projecting from the body 107′.

A bushing 122′ is also used for the metal element 104′.

Furthermore, two seals 123′ and 124′ are used in this embodiment, respectively associated with the pH sensor 101′ and with the metal element 104′.

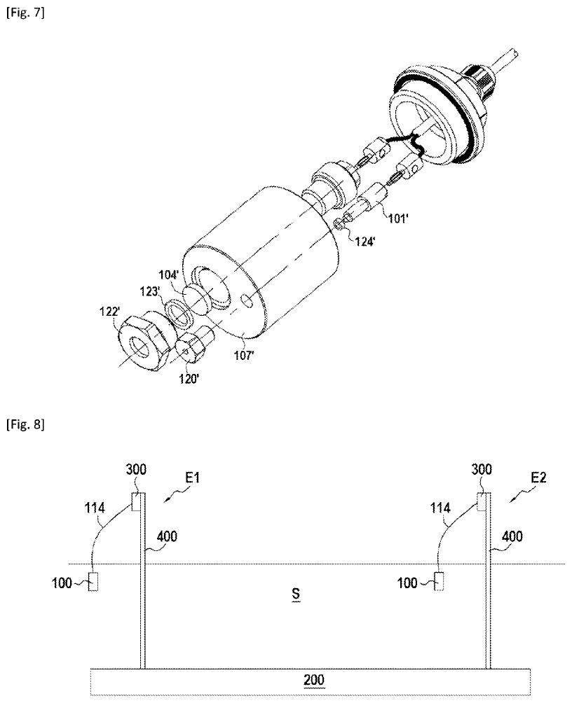

FIG. 8 illustrates a facility comprising a pipe 200 which is buried in the ground S. At a first location E1, a station 300 for measuring or tapping potential is installed in order to be connected to the pipe 200 by a cable 400. At a second location E2, a measurement station or potential tap 300 is also installed in order to be connected to the pipe 200 by a cable 400.

In general, these potential taps are used to measure the electrical potential of the pipe to which an electrical potential has been applied for the purposes of cathodic protection. These taps are arranged, for example, every 4 kilometres.

It is possible to use devices 100 equipped with pH sensors like those described above with reference to FIGS. 2 to 4 in order to measure the pH of the ground at the locations E1 and E2. Indeed, it is difficult to reach the pipe 200 in order to carry out a measurement. By contrast, the devices 100 can be inserted at a shallow depth into the ground so that they are in contact with the same environment as that of the pipe.

The potential taps 300 are used to apply electrical potentials applied directly on the pipe to the metal elements of the devices 100, in particular by using sleeved cables 114.

These measurements make it possible to monitor the change in pH in different regions, which then makes it possible to determine the value of the potential to be applied to the pipe 200 in order to avoid corrosion, for example by using a Pourbaix diagram.

1. A pH sensor device, intended to be inserted into the ground in order to be in contact with a region containing a fluid from the ground, for which fluid the pH is desired to be known, comprising:
a pH sensor comprising a surface comprising a polymer material capable of attracting protons and arranged so that the polymer material is in contact with said region containing a fluid when the device is inserted into the ground,
a metal element having a surface configured to be in contact with the region containing the fluid when the device is inserted into the ground,
electrical connection means connected at least to the metal element and configured to receive a cathodic-protection electrical potential for the metal element.

2. The device according to claim 1, wherein the polymer contains one or more amino groups.

3. The device according to claim 2, wherein the polymer is chosen in the group comprising polypyrrole and poly(3,4 ethylenedioxythiophene).

4. The device according to claim 1, comprising a body provided with at least a first opening configured to allow, when the device is inserted into the ground, the fluid contained in said region to be in contact with said surface comprising a polymer material and with said surface of the metal element, and at least one second opening configured to receive said electrical connection means.

5. The device according to claim 4, wherein said surface comprising a polymer material and said surface of the metal element are arranged recessed from an outer surface of the body where the openings are formed.

6. The device according to claim 4, comprising first means for maintaining the sealing arranged at the interface between, on the one hand, said surface comprising a polymer material and said surface of the metal element and, on the other hand, the body, in order to prevent the fluid contained in said region from penetrating inside the body.

7. The device according to claim 4, comprising second means for maintaining the sealing arranged at the interface between, on the one hand, the electrical connection means and, on the other hand, the body, in order to prevent the fluid contained in said region from penetrating inside the body.

8. The device according to claim 4, wherein the pH sensor and/or the metal element are detachable from the body.

9. The device according to claim 1, wherein said surface comprising a polymer material and said surface of the metal element are spaced apart by a strip having a thickness between 0.5 and 1.5 millimetres.

10. The device according to claim 1, wherein said surface of the metal element has an annular shape and said surface comprising a polymer material is arranged so as to be surrounded by said surface of the metal element.

11. The device according to claim 1, wherein said surface of the metal element has an area greater than or equal to at least 1 cm² and said surface comprising a polymer material has an area greater than or equal to at least 0.07 cm².

12. A method for measuring the pH of a fluid contained in a region of the ground, comprising:
inserting a device according to claim 1 into the ground so that said surface of the metal element and said surface comprising a polymer material are in contact with the fluid contained in a region of the ground,
applying a cathodic-protection electrical potential to the metal element via the electrical connection means, and
measuring the pH by means of the pH sensor.

13. A method for cathodic protection of a pipe comprising:
implementing the method for measuring the pH according to claim 12 for a region of the ground located above a pipe,
determining a new value of electrical potential to be applied to said pipe on the basis of the result of said pH measurement.
